(19) United States
(12) Patent Application Publication
(10) Pub. No.: US 2024/0408248 A1
(43) Pub. Date: Dec. 12, 2024

(54) GRANZYME B-TARGETING COMPLEX, RADIOPHARMACEUTICAL, PREPARATION METHOD THEREFOR AND USE THEREOF

(71) Applicant: PEKING UNIVERSITY, Beijing (CN)

(72) Inventors: Zhaofei Liu, Beijing (CN); Xing Yang, Beijing (CN); Yanpu Wang, Beijing (CN); Hongchuang Xu, Beijing (CN); Haoyi Zhou, Beijing (CN)

(21) Appl. No.: 18/709,848

(22) PCT Filed: Sep. 9, 2022
(86) PCT No.: PCT/CN2022/118207

§ 371 Date: May 14, 2024

(30) Foreign Application Priority Data
Nov. 18, 2021 (CN) 202111370138.7

(51) Int. Cl. A61K 51/04; A61P 35/00

(52) U.S. Cl. CPC A61K 51/0497; A61P 35/00

(57) ABSTRACT

The present invention belongs to the field of nuclear medicine and relates to a Granzyme B-targeting complex, a radiopharmaceutical, a preparation method therefor and a use thereof. A structure of the Granzyme B-targeting complex is shown in formula (I), where R is any one of a bifunctional chelating group or a derivative thereof for radionuclide labeling. The Granzyme B-targeting complex provided by the present invention can be prepared into the Granzyme B-targeting radiopharmaceutical through radionuclide labeling. The Granzyme B-targeting radiopharmaceutical provided is simple to prepare and has better pharmacokinetic characteristics and in vivo metabolic stability than other Granzyme B-targeting drugs. The expression level of Granzyme B in vivo can be monitored noninvasively by nuclear medicine imaging.

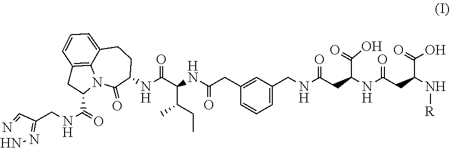

### FIELD OF TECHNOLOGY

The present invention belongs to the field of nuclear medicine, and specifically relates to a Granzyme B-targeting complex and preparation method thereof, a Granzyme B-targeting radiopharmaceutical and preparation method thereof, and their use in nuclear medicine imaging diagnosis and therapeutic imaging monitoring of related diseases such as tumors.

### BACKGROUND

Granzymes are a kind of serine protease. Human granzymes include five types: A, B, H, K, and M, which exist in cell granules released by cytotoxic T lymphocytes (CTL) and natural killer cells (NK). Granzyme B, as one of the main effector molecules of granzymes, can enter cells, mediate the activation of downstream caspase signaling pathways, induce cell DNA breakage, and thus lead to cell apoptosis. At the same time, Granzyme B can also cleave nuclear proteins, including NuMA and P, as well as DNA-PKcs et al., and promote and initiate nuclear apoptosis. Therefore, Granzyme B is one of the important markers for CTL or NK cells to be activated and exert cell killing effects.

In the field of tumor treatment, immunotherapy is considered to be one of the most ideal ways to eliminate tumors and prevent tumor metastasis and recurrence. At present, tumor immunotherapy has achieved a series of significant clinical breakthroughs, such as antibodies targeting immune checkpoints (such as CTLA-4, PD-1/PD-L1) and chimeric antigen receptors autologous T cell (CAR-T) therapy. However, the current effective rate of tumor immunotherapy is low. Taking anti-PD-1 immune checkpoint inhibition as an example, its effective rate is often less than 30%. Therefore, it is crucial to accurately predict the early efficacy of immunotherapy in order to effectively guide its precise treatment and improve treatment efficacy.

Nuclear medicine molecular imaging, represented by positron emission tomography (PET) and single-photon emission computed tomography (SPECT), provides effective technical means for noninvasive, dynamic, and quantitative imaging to monitor disease treatment. ¹⁸F-fluorodeoxyglucose (¹⁸F-FDG) is currently the most widely used PET imaging drug in clinical practice. ¹⁸F-FDG is an analog of glucose, and its biological behavior is similar to that of glucose. It reflects the occurrence and progression of diseases by monitoring the uptake of glucose by diseases. However, ¹⁸F-FDG lacks tumor specificity and is not a specific marker of active cells such as CTL or NK. It has obvious limitations in predicting and evaluating the efficacy of immunotherapy. Therefore, the development of new nuclear medicine imaging drugs for monitoring the efficacy of tumor immunotherapy has important clinical significance.

Granzyme B is a serine protease released by CTL and NK cells in the immune response process, and its expression level is closely related to the immune response. Therefore, if nuclear medicine imaging drugs with good Granzyme B target specificity, affinity, and in vivo metabolic characteristics can be developed, especially PET and SPECT drugs labeled with nuclides such as ⁶⁸Ga, ¹⁸F and ^{99m}Tc, they will play an important role in tumor immunotherapy and efficacy monitoring, with broad clinical application prospects.

In addition, Granzyme B also exhibits specific high expression in other lesions, such as autoimmune diseases and immune-induced myocarditis. The development of specific nuclear medicine imaging drugs targeting Granzyme B can also play an important role in the imaging diagnosis, treatment monitoring, and efficacy evaluation of such diseases.

### SUMMARY

Based on the above background, the present invention provides a novel Granzyme B-targeting complex, which is formed by coupling a Granzyme B-targeting molecule with a bifunctional chelating agent. This type of complex becomes a radiopharmaceutical for nuclear medicine imaging after being labeled with radionuclides. Nuclear medicine PET or SPECT imaging can achieve noninvasive and specific monitoring of Granzyme B expression, which is expected to be widely applied in tumor immunotherapy, and diagnosis and treatment monitoring of autoimmune diseases. The Granzyme B specific radiopharmaceutical of the present invention is easy to prepare and has good pharmacokinetic characteristics in vivo.

The first aspect of the present invention provides a Granzyme B-targeting complex, having a structure of formula (I):

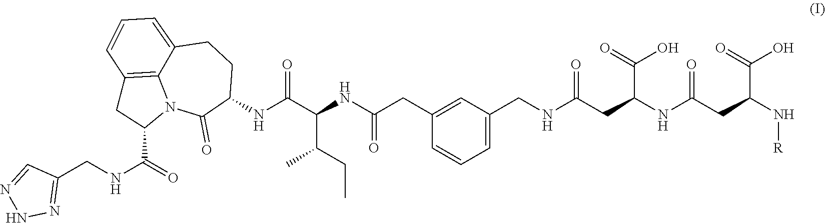

where R is any one of a bifunctional chelating group or a derivative thereof for radionuclide labeling.

According to the present invention, the bifunctional chelating group is a group formed by a bifunctional chelating agent, where preferably, the bifunctional chelating agent is DOTA, NOTA, HYNIC, MAG2, NODA, NODAGA, DOTP, TETA, ATSM, PTSM, EDTA, EC, HBEDCC, DTPA, BAPEN, Df, DFO, TACN, NO2A, NOTAM, CB-DO2A, Cyclen, DO3A, DO3AP, MAS3, MAG3, or isonitrile.

Further preferably, the R is any one of a group shown in formula (II), formula (III), formula (IV), formula (V), and formula (VI) or a derivative thereof,

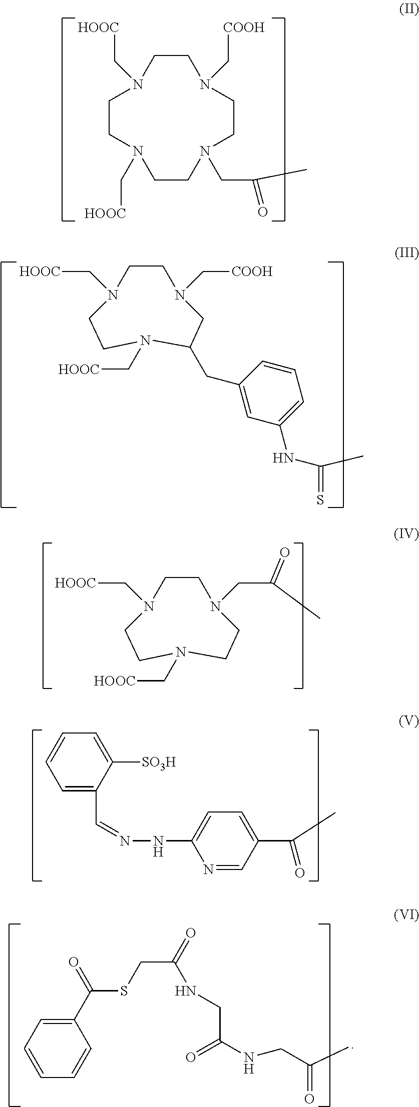

The second aspect of the present invention provides a Granzyme B-targeting radiopharmaceutical, obtained by labeling the complex with a radionuclide.

According to the present invention, the radionuclide can be a diagnostic radionuclide or a therapeutic radionuclide.

The diagnostic radionuclide is preferably at least one of ⁶⁸Ga, ⁶⁴Cu, ¹⁸F, ⁸⁶Y, ⁹⁰Y, ⁸⁹Zr, ¹¹¹In, ^{99m}Tc, ¹¹C, ¹²³I, ¹²⁵I, and ¹²⁴I.

The therapeutic radionuclide is preferably at least one of ¹⁷⁷Lu, ¹²⁵I, ¹³¹I, ²¹¹At, ¹¹¹In, ¹⁵³Sm, ¹⁸⁶Re, ¹⁸⁸Re, ⁶⁷Cu, ²¹²Pb, ²²⁵Ac, ²¹³Bi, ²¹²Bi, and ²¹²Pb.

According to some preferred embodiments of the present invention, the radionuclide is any one of ⁶⁸Ga, ⁶⁴Cu, ¹¹¹In, ¹⁸F, ⁸⁶Y, and ^{99m}Tc.

The third aspect of the present invention provides a preparation method of the Granzyme B-targeting complex, comprising following steps:
a. synthesizing a Granzyme B-targeting precursor according to a following solid-phase synthesis route; and

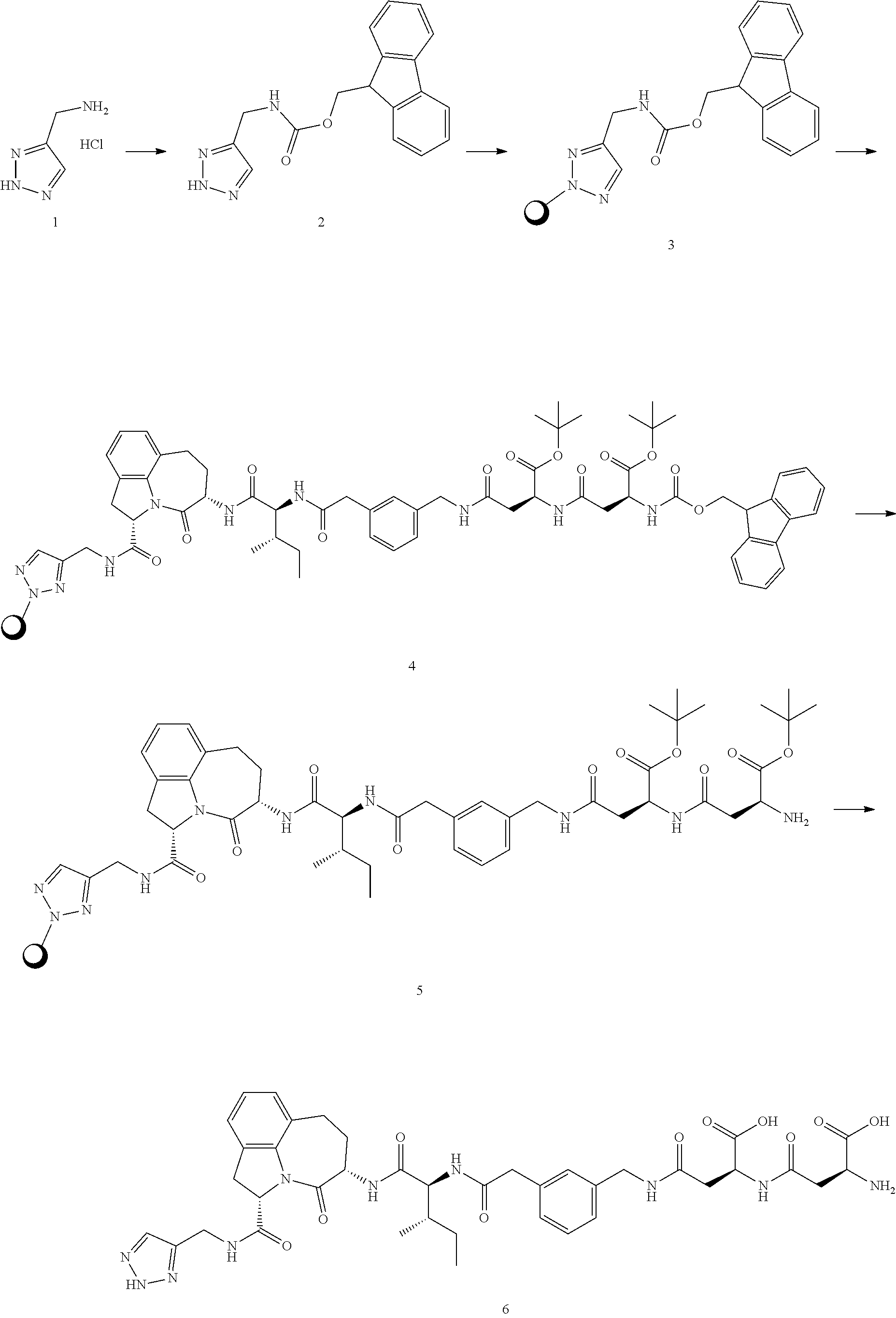

Reaction conditions: (a) DCM solution of Fmoc-NHS and DIPEA; (b) DMF/DCM solution of 2-chlorotriphenyl chloride resin and DIPEA; (c) DMF solution of 20% piperidine, and DMF solution of Fmoc-(2S,5S)-5-amino-1,2,4,5,6,7-hexahydroazepino[3,2,1-Hi]indole-4-one-2-carboxylic acid, HBTU, HOBt and DIPEA; (d) DMF solution of 20% piperidine, and DMF solution of Fmoc-L-isoleucine, HBTU, HOBt and DIPEA; (e) DMF solution of 20% piperidine, and DMF solution of Fmoc-(3-aminomethylphenyl) acetic acid, HBTU, HOBt and EIPEA; (f) DMF solution of 20% piperidine, and DMF solution of Fmoc-L-aspartate-1-tert-butyl ester, HBTU, HOBt and EIPEA; (g) DMF solution of 20% piperidine, and DMF solution of Fmoc-L-aspartate-1-tert-butyl ester, HBTU, HOBt and EIPEA; (h) DMF solution of 20% piperidine; (i) trifluoroacetic acid, water and triisopropylsilane; and (j) DIPEA and DMSO solution.

b. Coupling a Bifunctional Chelating Agent to the Granzyme B-Targeting Precursor.

The compounds used in the preparation process of the Granzyme B-targeting complex of the present invention can be commercially available or prepared through conventional organic synthesis methods.

Taking DOTA-NHS as an example, the bifunctional chelating agent is coupled to the Granzyme B-targeting precursor using a following synthetic route:

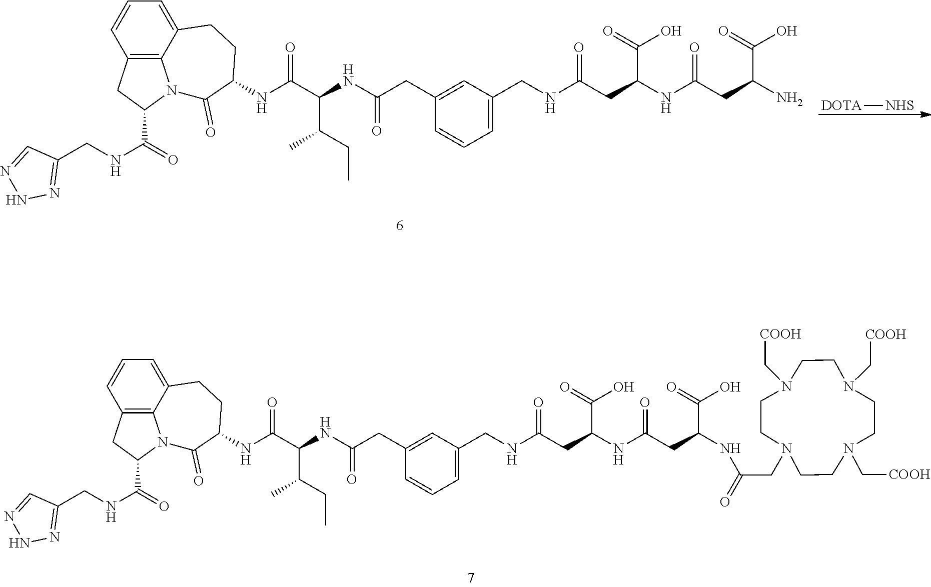

The fourth aspect of the present invention provides a preparation method of the Granzyme B-targeting radiopharmaceutical, including following steps: dissolving a Granzyme B-targeting complex in a radiolabeled buffer solution, then adding a different radionuclide for reaction, and after the reaction, separating and purifying a reaction solution through a Sep-Pak C18 chromatographic column to obtain the corresponding Granzyme B-targeting radiopharmaceutical.

According to a specific implementation of the present invention, when the complex is a DOTA-coupled complex and the radionuclide is any one of ⁶⁸Ga, ⁶⁴Cu, ¹¹¹In, and ⁸⁶Y, the preparation method includes following steps:
dissolving the DOTA-coupled complex in an acidic buffer solution, then adding ⁶⁸GaCl₃, ⁶⁴CuCl₂, ¹¹¹InCl₃, or ⁸⁶YCl₃, reacting for 10-60 min at 37° C., and then separating and purifying a reaction solution through a Sep-Pak C18 chromatographic column to obtain a corresponding ⁶⁸Ga-, ⁶⁴Cu-, ¹¹¹In-, or ⁸⁶Y-labeled complex.

According to a specific implementation of the present invention, when the complex is a NOTA-coupled complex and the radionuclide is any one of ⁶⁸Ga, ⁶⁴Cu and ¹⁸F, the preparation method includes following steps:
dissolving the NOTA-coupled complex in an acidic buffer solution, then adding ⁶⁸GaCl₃ or ⁶⁴CuCl₂, reacting for 10-30 min at 37° C., and after cooling, separating and purifying a reaction solution through a Sep-Pak C18 chromatographic column to obtain a corresponding ⁶⁸Ga- or ⁶⁴Cu-labeled complex; or
mixing a ¹⁸F ion with AlCl₃ in a sodium acetate buffer solution, reacting at room temperature for 2-8 min, then adding the NOTA-coupled complex to a mixture and reacting for 10-20 min under heating to 105-115° C., and after cooling, separating and purifying a reaction solution through a Sep-Pak C18 chromatographic column to obtain a corresponding ¹⁸F-labeled complex.

According to a specific implementation of the present invention, when the complex is MAG2-coupled complex or HYNIC-coupled complex and the radionuclide is ^{99m}Tc, the preparation method includes following steps:
dissolving the MAG2-coupled complex in an ammonium acetate and tartaric acid buffer solution, then adding Na^{99m}TcO₄, after mixing evenly, adding freshly prepared SnCl₂, then heating to 95-105° C. and reacting for 40-80 min, and after cooling, separating and purifying a reaction solution through a Sep-Pak C18 chromatographic column to obtain a corresponding ^{99m}Tc-labeled complex; or
after mixing the HYNIC-coupled complex, a TPPTS succinic acid buffer solution and a tricine succinic acid buffer solution, adding Na^{99m}TcO₄, then heating to 95-105° C. and reacting for 20-40 min, and after cooling, separating and purifying a reaction solution through a Sep-Pak C18 chromatographic column to obtain the corresponding ^{99m}Tc-labeled complex.

According to the present invention, preferably, after separation and purification, the purified product is diluted with normal saline and filtered aseptically to obtain an injection of each complex.

The fifth aspect of the present invention provides use of the Granzyme B-targeting complex or the Granzyme B-targeting radiopharmaceutical in preparation of a nuclear medicine imaging reagent.

The nuclear medicine imaging reagent is used for tumor imaging diagnosis and immunotherapy monitoring, for example. Specifically, for example, detecting the expression of Granzyme B during tumor immunotherapy (such as anti-PD-1/PD-L1, anti-CTLA-4, or CAR-T, CAR-NK anti-tumor therapy) to predict or monitor the efficacy of tumor immunotherapy; or used for other diseases that cause overexpression of Granzyme B, such as immune cardiomyopathy, Granzyme B-related side effects caused by immunotherapy, etc.

### The Beneficial Effects of the Present Invention

The Granzyme B-targeting complex provided by the present invention can be prepared into the Granzyme B-targeting radiopharmaceutical through radionuclide labeling. The Granzyme B-targeting radiopharmaceutical provided is simple to prepare and has better pharmacokinetic characteristics and in vivo metabolic stability than other Granzyme B-targeting drugs. The expression level of Granzyme B in vivo can be monitored noninvasively and quantitatively by nuclear medicine imaging.

The other features and advantages of the present invention will be explained in detail in the subsequent specific embodiments.

### BRIEF DESCRIPTION OF THE DRAWINGS

By providing a more detailed description of exemplary implementations of the present invention in conjunction with the accompanying drawings, the aforementioned and other objects, features, and advantages of the present invention will become more apparent.

FIG. 1 is a mass spectrogram of a DOTA-coupled Granzyme B-targeting complex.

FIG. 2 is a schematic diagram of a chemical structure of a ⁶⁸Ga-, ⁶⁴Cu-, ¹¹¹In-, or ⁸⁶Y-labeled DOTA-coupled Granzyme B-targeting complex.

FIG. 3 is a mass spectrogram of a NOTA-coupled Granzyme B-targeting complex.

FIG. 4 is a schematic diagram of a chemical structure of a ⁶⁸Ga- or ⁶⁴Cu-labeled NOTA-coupled Granzyme B-targeting complex.

FIG. 5 is a schematic diagram of a chemical structure of a ¹⁸F-labeled NOTA-coupled Granzyme B-targeting complex.

FIG. 6 is a mass spectrogram of a HYNIC-coupled Granzyme B-targeting complex.

FIG. 7 is a schematic diagram of a chemical structure of a ^{99m}Tc-labeled HYNIC-coupled Granzyme B-targeting complex.

FIG. 8 shows an experimental result of binding specificity between a ⁶⁸Ga-labeled DOTA complex and a coated Granzyme B.

FIG. 9 shows an in vitro stability result of a ⁶⁸Ga-labeled DOTA complex in PBS and FBS.

FIG. 10 shows a metabolic stability result of a ⁶⁸Ga-labeled DOTA complex in mice.

FIG. 11 is a PET imaging of a ⁶⁸Ga-labeled DOTA complex in tumor-bearing mice and its comparison with imaging characteristics of radiopharmaceuticals with similar structures.

FIG. 12 shows an experimental result of a correlation between an uptake value of a ⁶⁸Ga-labeled DOTA complex in MC38 tumors and an expression level of Granzyme B in tumors measured by in vitro Western blot.

FIG. 13 shows an experimental result of monitoring tumor anti-PD-1 immunotherapy by PET imaging with a ⁶⁸Ga-labeled DOTA complex.

FIG. 14 shows an experimental result of predicting pseudo-progression of tumor immunotherapy by PET imaging with a ⁶⁸Ga-labeled DOTA complex.

FIG. 15 shows an experimental result of PET imaging with a ⁶⁸Ga-labeled NOTA complex in tumor-bearing mice.

FIG. 16 shows an experimental result of PET imaging with a ¹⁸F-labeled NOTA complex in tumor-bearing mice.

FIG. 17 shows an experimental result of SPECT imaging with a ^{99m}Tc-labeled HYNIC complex in tumor-bearing mice.

### DESCRIPTION OF THE EMBODIMENTS

The preferred implementations of the present invention will be described in more detail below. Although the preferred implementations of the present invention are described below, it should be understood that the present invention can be implemented in various forms and should not be limited by the implementations described herein.

### Embodiment 1

### Synthesis of a Granzyme B-Targeting Complex.

The Granzyme B-targeting complex (Compound 7) was synthesized according to a following solid-phase synthesis route. R was any one of a bifunctional chelating group or a derivative thereof for radionuclide labeling. The bifunctional chelating group was formed by bifunctional chelating agent selected from DOTA, NOTA, HYNIC, or MAG2.

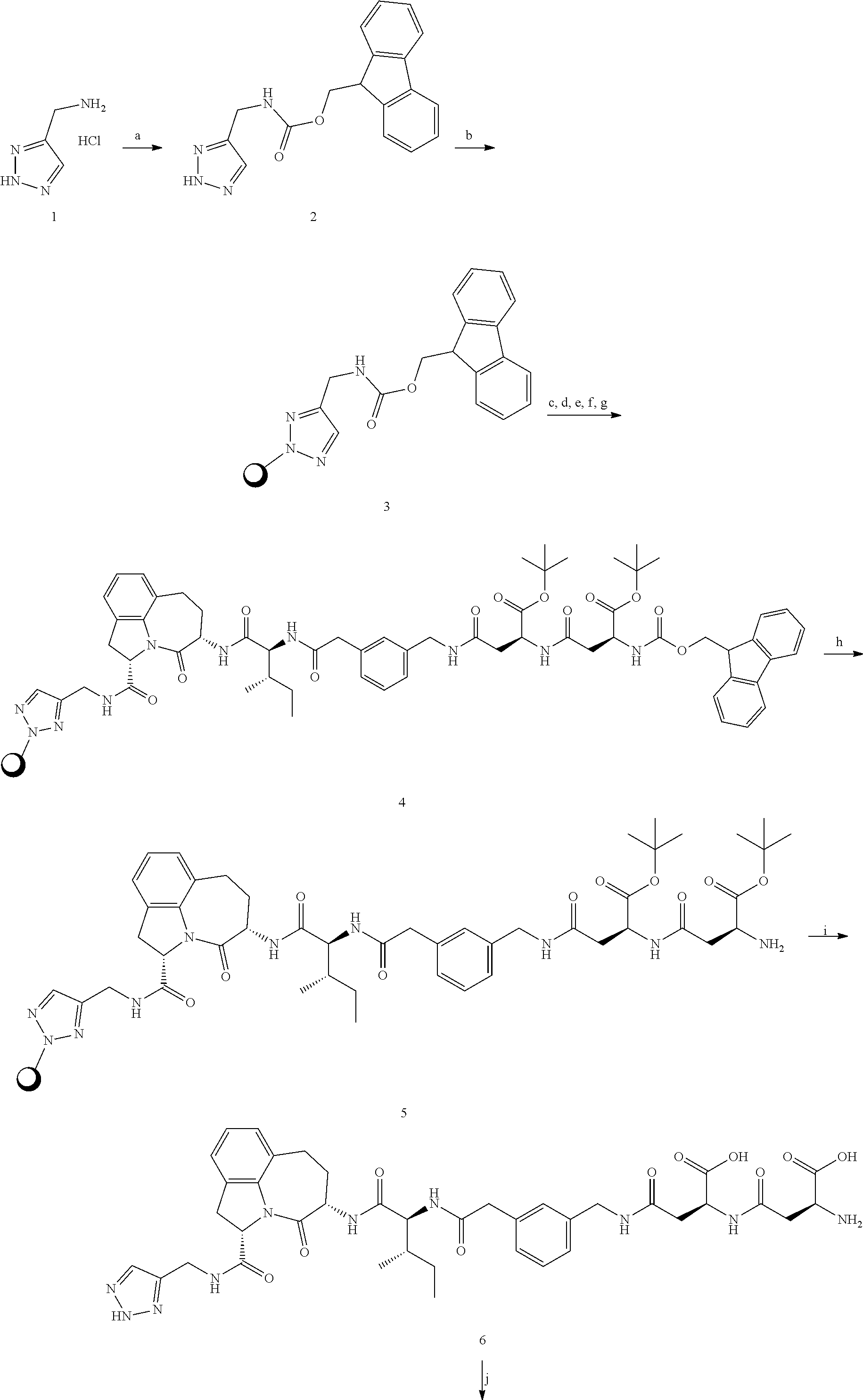

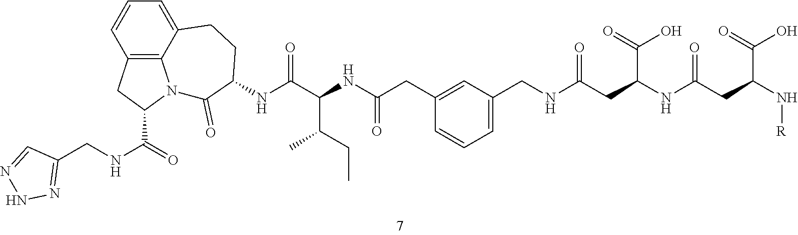

The specific synthesis steps were as follows:

Reaction conditions: (a) DCM solution of Fmoc-NHS and DIPEA; (b) DMF/DCM solution of 2-chlorotriphenyl chloride resin and DIPEA; (c) DMF solution of 20% piperidine, and DMF solution of Fmoc-(2S,5S)-5-amino-1,2,4,5,6,7-hexahydroazepino[3,2,1-Hi]indole-4-one-2-carboxylic acid, HBTU, HOBt and DIPEA; (d) DMF solution of 20% piperidine, and DMF solution of Fmoc-L-isoleucine, HBTU, HOBt and DIPEA; (e) DMF solution of 20% piperidine, and DMF solution of Fmoc-(3-aminomethylphenyl) acetic acid, HBTU, HOBt and EIPEA; (f) DMF solution of 20% piperidine, and DMF solution of Fmoc-L-aspartate-1-tert-butyl ester, HBTU, HOBt and EIPEA; (g) DMF solution of 20% piperidine, and DMF solution of Fmoc-L-aspartate-1-tert-butyl ester, HBTU, HOBt and EIPEA; (h) DMF solution of 20% piperidine; (i) trifluoroacetic acid, water and triisopropylsilane; and (j) DIPEA and DMSO solution.

Synthesis of Compound 2: 1H-[1, 2, 3] thiazol-4-ylmethylamine hydrochloride (1,200.00 mg, 2.04 mmol) and 9-fluorenomethyl-N-succinimidyl carbonate (825.77 mg, 2.45 mmol) were taken in a 50 mL round-bottom flask, 10 mL DCM was added to dissolve them, and DIPEA (526.32 mg, 4.08 mmol) was added. Electromagnetic stirring was performed at room temperature. The reaction was stopped after 4 h. Vacuum distillation was performed, 100 mL of DCM was added to the mixture and washed with water (50 mL×2), the organic phase was collected and dried by anhydrous magnesium sulfate, then dichloromethane was removed under pressure, and the product was purified by a silica gel column to obtain White solid 2.

Synthesis of Resin 3: 2-chlorotriphenyl chloride resin (1.00 g) was placed in a solid-phase synthesis tube, 2 mL dichloromethane (DCM) was added for swelling, which was repeated three times for 5 min each, and then N,N-dimethylformamide (DMF) was used for washing three times for 5 min each. Compound 2 (96.00 mg, 0.30 mmol) was dissolved in a mixed solvent of DCM and DMF, DIPEA (78 mg, 0.60 mmol) was added, the above mixture was added to a solid-phase synthesis tube, electromagnetic stirring was performed at room temperature, and the reaction was stopped after 2 h; 2 mL dichloromethane (DCM) was used for washing, which was repeated three times for 5 min each; resin was sealed using a mixed solvent of 7 mL (DCM:MeOH:DIPEA=10 mL:10 mL:1 mL), which was repeated three times for 5 min each; and 2 mL dichloromethane (DCM) was used for washing, which was repeated three times for 5 min each, and the solvent was removed under reduced pressure to obtain Yellow resin 3.

The synthesis of Compound 4: the coupling of amino acids was carried out according to the standard Fmoc solid-phase synthesis method. A certain mass of Resin 3 (0.25 mmol) was taken in a 10 mL solid-phase synthesis tube, 2 mL dichloromethane (DCM) was added for swelling, which was repeated three times for 5 min each, and then N,N-dimethylformamide (DMF) was used for washing three times for 5 min each. The amino protective group Fmoc was removed using a DMF solution containing 20% piperidine (v/v). The specific operation was to react with 2 mL of a DMF solution of 20% piperidine for 2 min, 10 min and 10 min, followed by 3-5 washes with 2 mL DMF for 2 min each. Compared with the resin (0.02 mmol), 3 times the chemical amount of Fmoc amino acid was activated by 3.6 times the chemical amount of HBTU in the presence of 7.2 times the chemical amount of DIPEA, then added to the synthesis tube, and reacted under electromagnetic stirring for 1 h.

Synthesis of Compound 5: the amino protective group Fmoc was removed using a DMF solution containing 20% piperidine (v/v) as described in the above method.

Synthesis of Compound 6: the dissociation of the compound from the resin and the removal of the tert-butyl ester were completed by using 5 mL trifluoroacetic acid/triisopropylsilane/water (95:2.5:2.5, v/v/v) and stirring for 2 h, and the resin was washed with 2 mL trifluoroacetic acid, all filtrates were collected, trifluoroacetic acid was removed under reduced pressure, and then the crude product was obtained through reverse preparation by HPLC and lyophilization.

Synthesis of Compound 7: A certain mass of Compound 6 (5 μmol) was taken and dissolved in 500 μL of DMSO, followed by adding 10 molar ratios of the bifunctional chelating agent-NHS or bifunctional chelating agent-p-SCN-Bn, and DIPEA (10 μmol). After mixing evenly and reacting at room temperature for 2 h, the crude product was purified by HPLC and lyophilized.

### Embodiment 2

Synthesis of a DOTA-coupled complex and preparation of a corresponding radiopharmaceutical by labeling it with any one of radionuclides ⁶⁸Ga, ⁶⁴Cu, ¹¹¹In, and ⁸⁶Y. Specifically, it included the following steps:

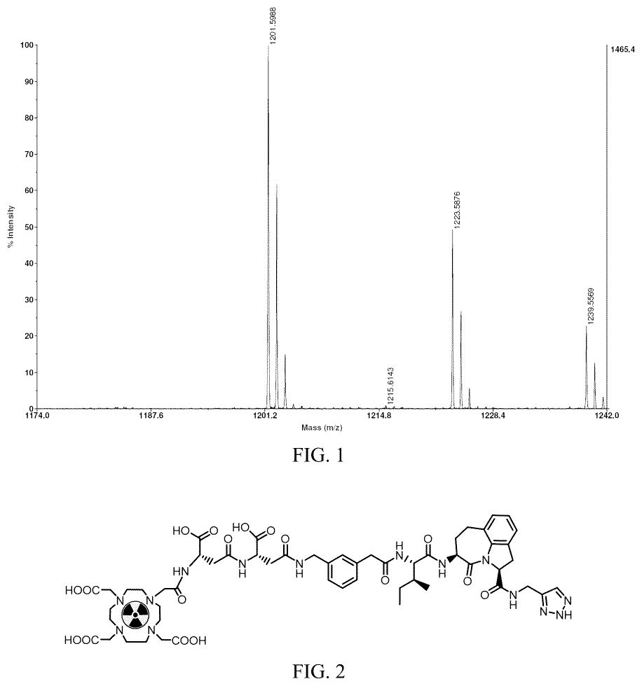

5 μmol of Compound 6 was taken and dissolved in 500 μL of DMSO, followed by adding 50 μmol of DOTA-NHS and 10 μmol of DIPEA. After mixing evenly and reacting at room temperature for 2 h, the crude product was purified by HPLC and lyophilized to obtain the DOTA-coupled Granzyme B-targeting complex. Its mass spectrometry characterization is shown in FIG. 1.

10 nmol of the DOTA-coupled complex was dissolved in 300 μL of 0.1 M sodium acetate buffer solution (pH=5.5); then 185 MBq of ⁶⁸GaCl₃, ⁶⁴CuCl₂, ¹¹¹InCl₃, or ⁸⁶YCl₃ was added for reaction for 30 min at 37° C., then the reaction solution was separated and purified on a Sep-Pak C18 chromatographic column, and the purified product was diluted with normal saline and filtered aseptically to obtain an injection of the corresponding ⁶⁸Ga-, ⁶⁴Cu-, ¹¹¹In-, or ⁸⁶Y-labeled complex. The schematic diagram of its chemical structure is shown in FIG. 2.

### Embodiment 3

Synthesis of a NOTA-coupled complex and preparation of a corresponding radiopharmaceutical by labeling it with any one of radionuclides ⁶⁸Ga, ⁶⁴Cu or ¹⁸F. Specifically, it included the following steps:

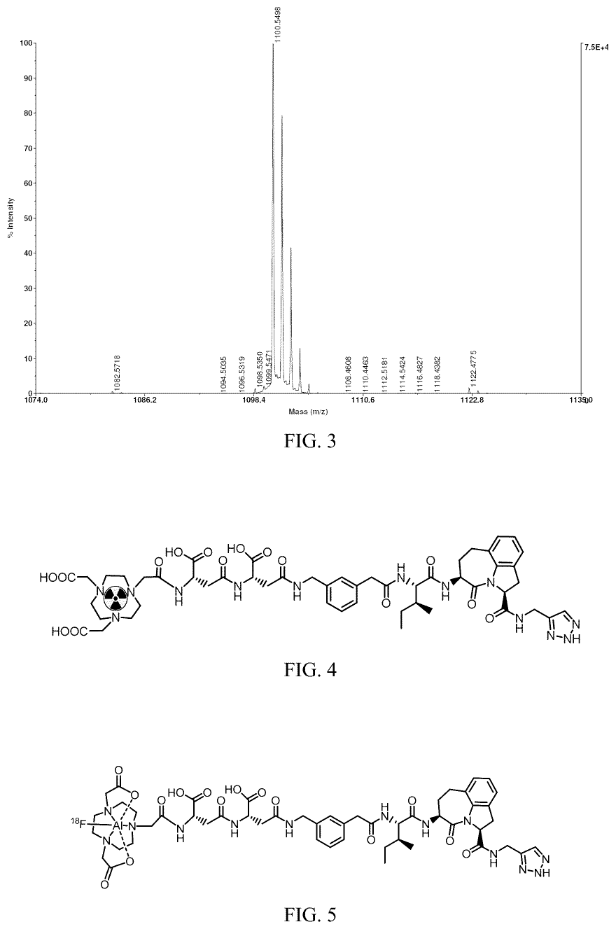

5 μmol of Compound 6 was taken and dissolved in 500 μL of DSMO, followed by adding 50 μmol of NOTA-NHS and 10 μmol of DIPEA. After mixing evenly and reacting at room temperature for 2 h, the crude product was purified by HPLC and lyophilized to obtain the NOTA-coupled Granzyme B-targeting complex. Its mass spectrometry characterization is shown in FIG. 3.

⁶⁸Ga or ⁶⁴Cu radioactive labeling: 10 nmol of the NOTA-coupled complex was dissolved in 300 μL of 0.1 M sodium acetate buffer solution (pH=5.5); then 185 MBq of ⁶⁸GaCl₃ or ⁶⁴CuCl₂ was added for reaction for 15 min at 37° C., after cooling, the reaction solution was separated and purified by a Sep-Pak C18 chromatographic column, and the purified product was diluted with normal saline and filtered aseptically to obtain an injection of the corresponding ⁶⁸Ga- or ⁶⁴Cu-labeled complex. The schematic diagram of its chemical structure is shown in FIG. 4.

¹⁸F radioactive labeling: 740 MBq of ¹⁸F ions were mixed with 24 nmol AlCl₃ in 100 μL of sodium acetate buffer (0.1 M, pH=4.0) and reacted for 5 min at room temperature. Subsequently, 40 nmol of the NOTA-coupled complex was added to the mixture and reacted at 110° C. for 15 min. After cooling, the reaction solution was separated and purified by a Sep-Pak C18 chromatographic column, and the purified product was diluted with normal saline and filtered aseptically to obtain an injection of the corresponding ¹⁸F-labeled complex. The schematic diagram of its chemical structure is shown in FIG. 5.

### Embodiment 4

Synthesis of a HYNIC-coupled complex and preparation of a corresponding radiopharmaceutical by labeling it with a radionuclide ^{99m}Tc. Specifically, it included the following steps:

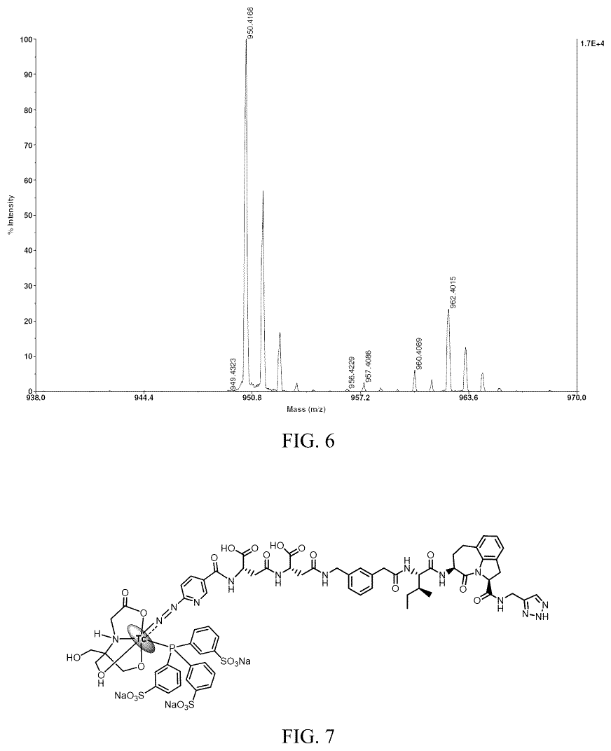

5 μmol of Compound 6 was taken and dissolved in 500 μL of DMSO, followed by adding 50 μmol of HYNIC-NHS and 10 μmol of DIPEA. After mixing evenly and reacting at room temperature for 2 h, the crude product was purified by HPLC and lyophilized to obtain the HYNIC-coupled Granzyme B-targeting complex. Its mass spectrometry characterization is shown in FIG. 6.

After mixing 10 nmol of the HYNIC-coupled complex, 100 μL of TPPTS (100 μg/μL in 25 mM succinic acid buffer solution) and tricine (100 mg/mL in 25 mM succinic acid buffer solution), 370 MBq of Na^{99m}TcO₄ was added, followed by heating to 100° C. for 30 min. After cooling, the reaction solution was separated and purified by a Sep-Pak C18 chromatographic column, and the purified product was diluted with normal saline and filtered aseptically to obtain an injection of the corresponding ^{99m}Tc-labeled complex. The schematic diagram of its chemical structure is shown in FIG. 7.

### Embodiment 5

### In Vitro Granzyme B Binding Specificity of a ⁶⁸Ga-Labeled DOTA Complex.

Granzyme B was coated in an ELISA plate, and 7.4 KBq of the ⁶⁸Ga-labeled DOTA complex was added to the coated plate. After reaction for 0.5 h at room temperature, the plate was washed, and the binding amount of the ⁶⁸Ga-labeled DOTA complex in the plate was determined by a gamma counter. The result is illustrated in FIG. 8, showing that the binding of the ⁶⁸Ga-labeled DOTA complex to Granzyme B was significantly higher than that of the control group, confirming the in vitro Granzyme B binding specificity of the ⁶⁸Ga-labeled DOTA complex.

### Embodiment 6

### In Vitro Stability of a ⁶⁸Ga-Labeled DOTA Complex.

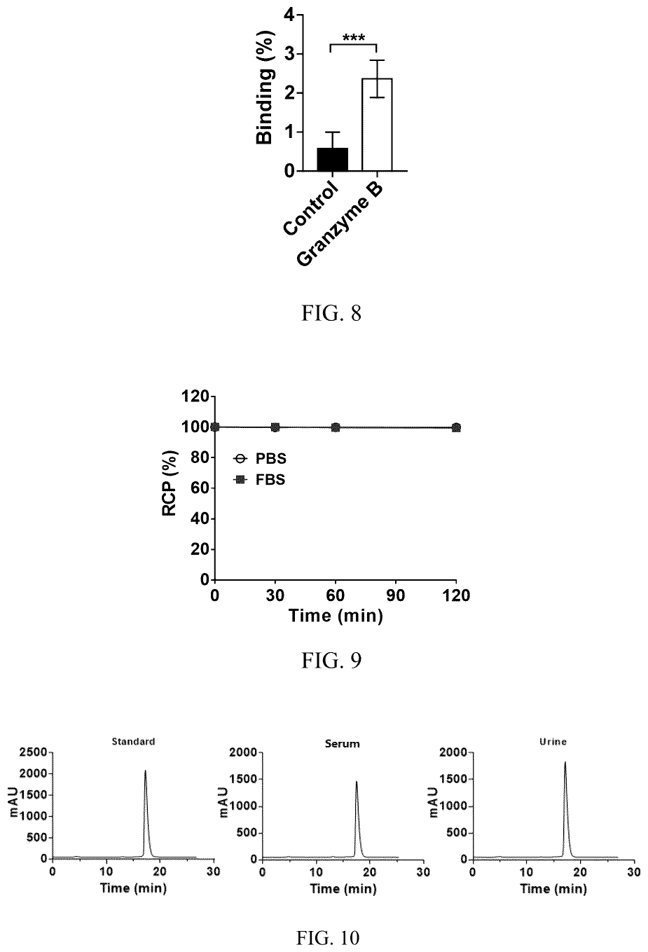

3.7 MBq of the ⁶⁸Ga-labeled DOTA complex was dissolved in PBS or 10% fetal bovine serum (FBS) and incubated at room temperature for 0 h, 0.5 h, 1 h, and 2 h, respectively. Afterwards, the radiochemical purity (RCP) was determined by radioactive thin layer chromatography. The result is illustrated in FIG. 9, showing that the ⁶⁸Ga-labeled DOTA complex exhibited good stability in both PBS and FBS.

### Embodiment 7

### In Vivo Metabolic Stability of a ⁶⁸Ga-Labeled DOTA Complex.

BALB/c mice were injected with 37 MBq of the ⁶⁸Ga-labeled DOTA complex via the tail vein. The serum and urine were taken 0.5 hours after injection. After centrifugation, the supernatant was diluted with 50% acetonitrile in an aqueous solution. The stability was analyzed by HPLC after filtration with 0.22 μm membrane. The result is illustrated in FIG. 10, showing that the ⁶⁸Ga-labeled DOTA complex maintained its original form in both the urine and serum, indicating its excellent in vivo metabolic stability.

### Embodiment 8

PET imaging of a ⁶⁸Ga-labeled DOTA complex in tumor-bearing mice and its comparison with imaging characteristics of radiopharmaceuticals with similar structures.

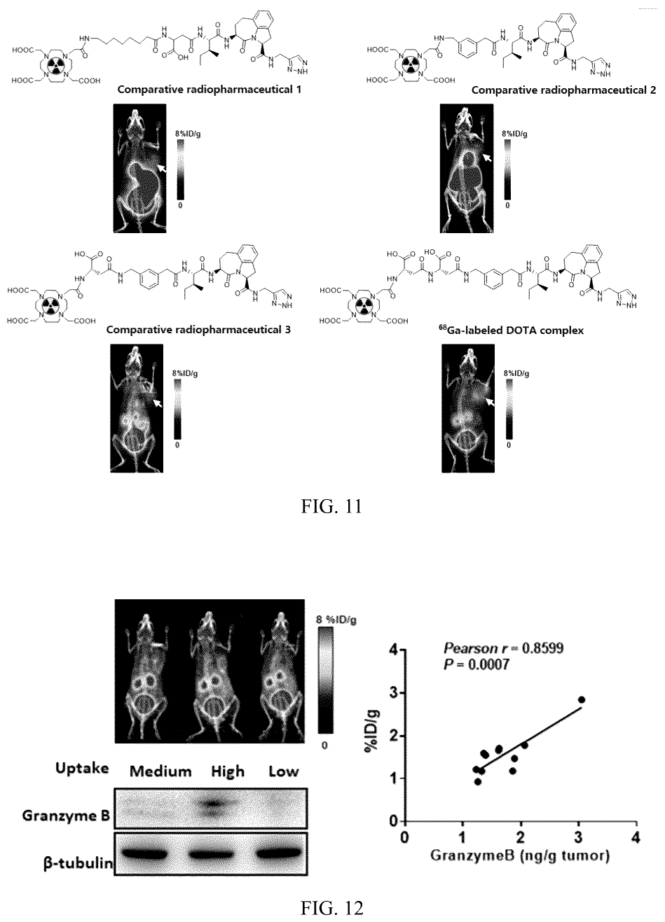

In order to compare the in vivo nuclear medicine imaging advantages of the Granzyme B-targeting complex of the present invention, three Granzyme B-targeting complexes with similar structures are prepared by a similar synthesis method. All of them were labeled with ⁶⁸Ga after DOTA coupling to obtain the corresponding Granzyme B-targeting radioactive compounds. 7.4 MBq of the freshly prepared ⁶⁸Ga-labeled complex was taken and injected into female C57 mice bearing MC38 tumors via the tail vein. After injection for 0.5 h, the animals were anesthetized with isoflurane for PET/CT imaging. The result is illustrated in FIG. 11, showing that the ⁶⁸Ga-labelled DOTA complex of the present invention had optimal tumor Granzyme B uptake and low normal tissue (e.g. gastrointestinal tract) uptake compared with the other three comparative radiopharmaceuticals. It was indicated that the Granzyme B-targeting complex of the present invention has the optimal nuclear medicine imaging characteristics in vivo.

### Embodiment 9

### Quantitative Detection of Expression Level of Tumor Granzyme B by PET Imaging of the ⁶⁸Ga-Labeled DOTA Complex.

7.4 MBq of the freshly prepared ⁶⁸Ga-labeled complex was taken and injected into female C57 mice bearing MC38 tumors in the right axilla via the tail vein. After injection for 0.5 h, the animals were anesthetized with isoflurane for PET/CT imaging. After the imaging is completed, the mice were sacrificed and their tumor tissue was taken. The expression level of Granzyme B in the tumor was determined by Western blot after the tumor tissue was ground to extract the protein. The result is illustrated in FIG. 12, showing that the uptake value of the ⁶⁸Ga-labeled DOTA complex in MC38 tumors had a good linear correlation with the expression level of Granzyme B in the tumors determined by Western blot in vitro. It was indicated that nuclear medicine imaging with the Granzyme B-targeting radiopharmaceutical described in the present invention can noninvasively determine the expression level of Granzyme B and dynamically monitor its expression changes.

### Embodiment 10

Monitoring tumor anti-PD-1 immunotherapy by PET imaging of ⁶⁸Ga-labeled DOTA complex. C57 mice bearing MC38 tumors in the right axilla were intraperitoneally injected with 200 μg of the anti-PD-1 antibody 3 times on day 0, day 3 and day 6, respectively. On day 9, tumor-bearing mice were injected with 7.4 MBq of the freshly prepared ⁶⁸Ga-labeled complex via the tail vein. After injection for 0.5 h, the animals were anesthetized with isoflurane for PET/CT imaging. The change of the tumor size in MC38 tumor-bearing mice was measured by a vernier caliper. The result is illustrated in FIG. 13, showing that the uptake level of the ⁶⁸Ga-labeled DOTA complex in the tumor could well distinguish whether the tumor was responsive to anti-PD-1 immunotherapy. It was indicated that the therapeutic effect of tumor immunotherapy could be predicted by noninvasive monitoring of the expression level of Granzyme B through the Granzyme B-targeting radiopharmaceutical nuclear medicine imaging in the present invention.

### Embodiment 11

### Identifying Pseudo-Progression of Tumor Immunotherapy by PET Imaging of ⁶⁸Ga-Labeled DOTA Complex.

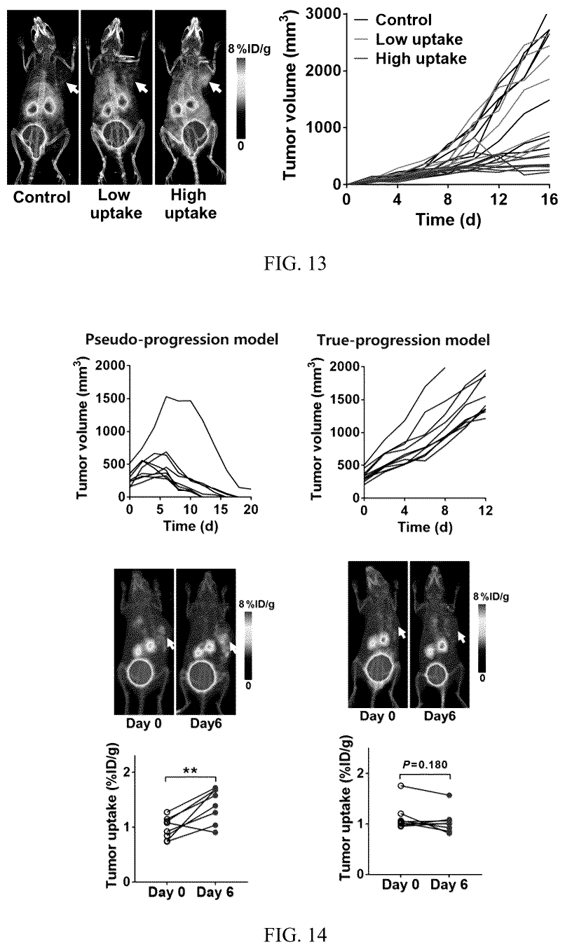

MC38 and 4T1 tumor cells were inoculated subcutaneously in the right axilla of C57 and BALB/c mice, respectively, to establish immunotherapy pseudo-progression and true-progression mouse models. 200 μg of the anti-PD-1 antibody and anti-CTLA-4 antibody were injected intraperitoneally for a total of 3 times on days 0, 3 and 6, respectively. On day 12, tumor-bearing mice were injected with 7.4 MBq of the freshly prepared ⁶⁸Ga-labeled complex via the tail vein. After injection for 0.5 h, the animals were anesthetized with isoflurane for PET/CT imaging. The changes of the tumor size in MC38 and 4T1 tumor-bearing mice were measured by a vernier caliper. The result is illustrated in FIG. 14, showing that in the MC38 pseudo-progression mouse tumor model, the tumor uptake of the ⁶⁸Ga-labeled complex at day 6 was significantly higher than that at day 0. In the 4T1 true-progression mouse tumor model, there was no significant difference in the tumor uptake of the ⁶⁸Ga-labeled complex on day 6 and day 0. It was indicated that the ⁶⁸Ga-labeled complex could monitor the expression of Granzyme B in tumors during immunotherapy, and predict the true- and pseudo-progression of tumor immunotherapy by reflecting the activation status of T cells.

### Embodiment 12

PET Imaging of Tumor Granzyme B with a ⁶⁸Ga-Labeled NOTA Complex.

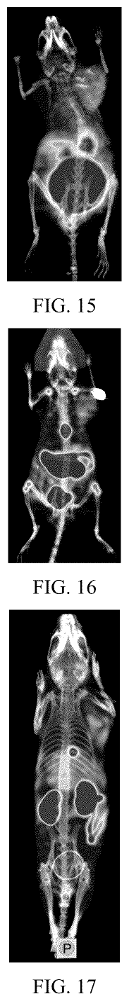

7.4 MBq of the freshly prepared ⁶⁸Ga-labeled NOTA complex was taken and injected into female C57 mice bearing MC38 tumors in the right axilla via the tail vein. After injection for 0.5 h, the animals were anesthetized with isoflurane for PET/CT imaging. The result is illustrated in FIG. 15, showing that the ⁶⁸Ga-labeled NOTA complex also had a good uptake value in MC38 tumors. It was indicated that the replacement of different bifunctional chelating agents for the Granzyme B-targeting radiopharmaceutical in the present invention did not affect the Granzyme B-targeting characteristic of the Granzyme B-targeting radiopharmaceutical.

### Embodiment 13

7.4 MBq of the freshly prepared ¹⁸F-labeled NOTA complex was taken and injected into female C57 mice bearing MC38 tumors in the right axilla via the tail vein. After injection for 0.5 h, the animals were anesthetized with isoflurane for PET/CT imaging. The result is illustrated in FIG. 16, showing that the ¹⁸F-labeled NOTA complex also had a good uptake value in MC38 tumors. It was indicated that the replacement of different bifunctional chelating agents and the replacement of different radionuclides for the Granzyme B-targeting radiopharmaceutical in the present invention did not affect the Granzyme B-targeting characteristic of the Granzyme B-targeting radiopharmaceutical.

### Embodiment 14

SPECT imaging of tumor Granzyme B with a ^{99m}Tc-labeled HYNIC complex. 18.5 MBq of the freshly prepared ^{99m}Tc-labeled HYNIC complex was taken and injected into female C57 mice bearing MC38 tumors in the right axilla via the tail vein. After injection for 0.5 h, the animals were anesthetized with isoflurane for SPECT/CT imaging. The result is illustrated in FIG. 17, showing that the ^{99m}Tc-labeled HYNIC complex also had a good uptake value in MC38 tumors. It was indicated that the Granzyme B-targeting radiopharmaceutical in the present invention could also be used for Granzyme B-targeting specific nuclear medicine imaging after replacing different bifunctional chelating agents and performing different radionuclide labeling.

Embodiments of the present invention have been described above and the above description is exemplary, not exhaustive, and is not limited to the disclosed embodiments. Many modifications and alterations are obvious to those of ordinary skill in the art, without departing from the scope and spirit of the illustrated embodiments.

1. A Granzyme B-targeting complex, having a structure of formula (I): 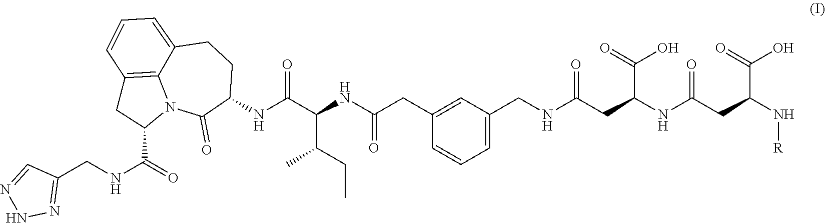
wherein R is any one of a bifunctional chelating group or a derivative thereof for radionuclide labeling.

2. The Granzyme B-targeting complex according to claim 1, wherein the bifunctional chelating group is a group formed by a bifunctional chelating agent, wherein the bifunctional chelating agent is DOTA, NOTA, HYNIC, MAG2, NODA, NODAGA, DOTP, TETA, ATSM, PTSM, EDTA, EC, HBEDCC, DTPA, BAPEN, Df, DFO, TACN, NO2A, NOTAM, CB-DO2A, Cyclen, DO3A, DO3AP, MAS3, MAG3, or isonitrile.

3. The Granzyme B-targeting complex according to claim 2, wherein the R is any one of a group shown in formula (II), formula (III), formula (IV), formula (V), and formula (VI) or a derivative thereof,

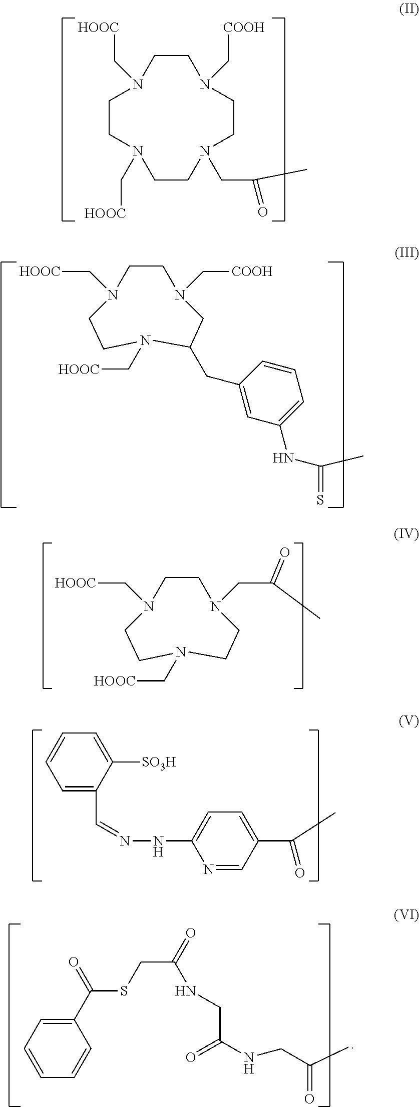

4-10. (canceled)

11. The Granzyme B-targeting complex according to claim 1, wherein the preparation method of the Granzyme B-targeting complex comprises following steps:
a. synthesizing a Granzyme B-targeting precursor according to a following solid-phase synthesis route; and 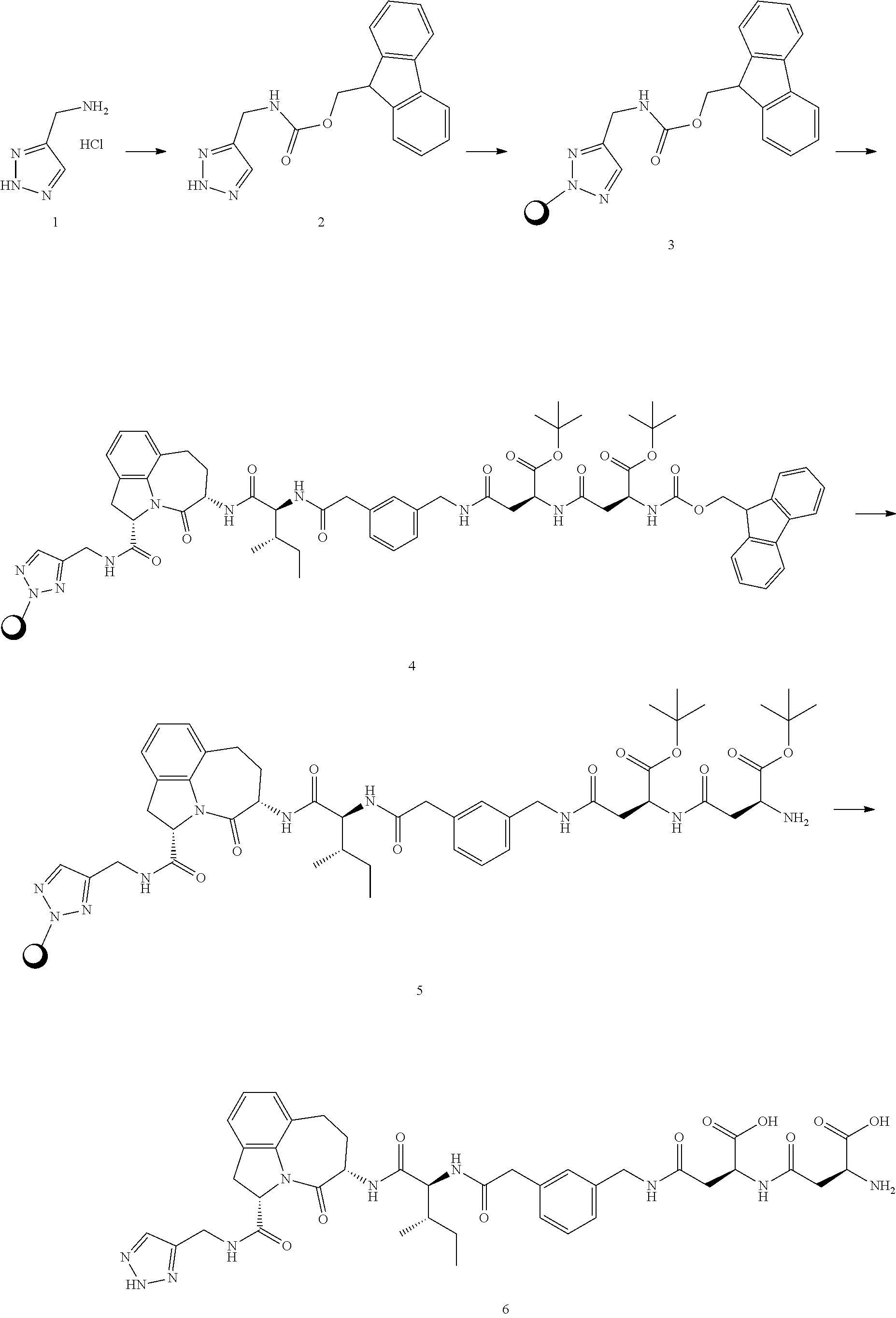
b. coupling a bifunctional chelating agent to the Granzyme B-targeting precursor.

12. A Granzyme B-targeting radiopharmaceutical, obtained by labeling the complex according to claim 1 with a radionuclide, wherein the radionuclide is a diagnostic radionuclide or a therapeutic radionuclide, wherein
the diagnostic radionuclide is preferably at least one of ⁶⁸Ga, ⁶⁴Cu, ¹⁸F, ⁸⁶Y, ⁹⁰Y, ⁸⁹Zr, ¹¹¹In, ^{99m}Tc, ¹¹C, ¹²³I, ¹²⁵I, and ¹²⁴I; and
the therapeutic radionuclide is preferably at least one of ¹⁷⁷Lu, ¹²⁵I, ¹³¹I, ²¹¹At, ¹¹¹In, ¹⁵³Sm, ¹⁸⁶Re, ¹⁸⁸Re, ⁶⁷Cu, ²¹²Pb, ²²⁵Ac, ²¹³Bi, ²¹²Bi, and ²¹²Pb.

13. The Granzyme B-targeting radiopharmaceutical according to claim 12, wherein the preparation method of the Granzyme B-targeting radiopharmaceutical comprises following steps: dissolving a Granzyme B-targeting complex in a radiolabeled buffer solution, then adding a different radionuclide for reaction, and after the reaction, separating and purifying a reaction solution through a Sep-Pak C18 chromatographic column to obtain the corresponding Granzyme B-targeting radiopharmaceutical.

14. The Granzyme B-targeting radiopharmaceutical according to claim 13, wherein the complex is a DOTA-coupled complex, the radionuclide is any one of ⁶⁸Ga, ⁶⁴Cu, ¹¹¹In, and ⁸⁶Y, and the preparation method comprises following steps:
dissolving the DOTA-coupled complex in an acidic buffer solution, then adding ⁶⁸GaCl₃, ⁶⁴CuCl₂, ¹¹¹InCl₃, or ⁸⁶YCl₃, reacting for 10-60 min at 37° C., and then separating and purifying a reaction solution through a Sep-Pak C18 chromatographic column to obtain a corresponding ⁶⁸Ga-, ⁶⁴Cu-, ¹¹¹In-, or ⁸⁶Y-labeled complex.

15. The Granzyme B-targeting radiopharmaceutical according to claim 13, wherein the complex is a NOTA-coupled complex, the radionuclide is any one of ⁶⁸Ga, ⁶⁴Cu and ¹⁸F, and the preparation method comprises following steps:
dissolving the NOTA-coupled complex in an acidic buffer solution, then adding ⁶⁸GaCl₃ or ⁶⁴CuCl₂, reacting for 10-30 min at 37° C., and after cooling, separating and purifying a reaction solution through a Sep-Pak C18 chromatographic column to obtain a corresponding ⁶⁸Ga- or ⁶⁴Cu-labeled complex; or
mixing a ¹⁸F ion with AlCl₃ in a sodium acetate buffer solution, reacting at room temperature for 2-8 min, then adding the NOTA-coupled complex to a mixture and reacting for 10-20 min under heating to 105-115° C., and after cooling, separating and purifying a reaction solution through a Sep-Pak C18 chromatographic column to obtain a corresponding ¹⁸F-labeled complex.

16. The Granzyme B-targeting radiopharmaceutical according to claim 13, wherein the complex is an MAG2-coupled complex or a HYNIC-coupled complex, the radionuclide is ^{99m}Tc, and the preparation method comprises following steps:
dissolving the MAG2-coupled complex in an ammonium acetate and tartaric acid buffer solution, then adding Na^{99m}TcO₄, after mixing evenly, adding freshly prepared SnCl₂, then heating to 95-105° C. and reacting for 40-80 min, and after cooling, separating and purifying a reaction solution through a Sep-Pak C18 chromatographic column to obtain a corresponding ^{99m}Tc-labeled complex; or
after mixing the HYNIC-coupled complex, a TPPTS succinic acid buffer solution and a tricine succinic acid buffer solution, adding Na^{99m}TcO₄, then heating to 95-105° C. and reacting for 20-40 min, and after cooling, separating and purifying a reaction solution through a Sep-Pak C18 chromatographic column to obtain the corresponding ^{99m}Tc-labeled complex.

17. Use of the Granzyme B-targeting radiopharmaceutical according to claim 12 in preparation of a nuclear medicine imaging reagent, wherein preferably, the nuclear medicine imaging reagent is used for tumor imaging diagnosis and immunotherapy monitoring.
